(19) United States
(12) Patent Application Publication
(10) Pub. No.: US 2016/0174930 A1
(43) Pub. Date: Jun. 23, 2016

(54) IMAGING ARRANGEMENT AND METHOD FOR POSITIONING A PATIENT IN AN IMAGING MODALITY

(71) Applicant: Siemens Aktiengesellschaft, Munchen (DE)

(72) Inventors: Christoph BRAUN, Rosenheim (DE); Johann UEBLER, Nuernberg (DE)

(21) Appl. No.: 14/964,667

(22) Filed: Dec. 10, 2015

(30) Foreign Application Priority Data
Dec. 22, 2014 (DE) 102014226756.0

(51) Int. Cl. A61B 6/00; A61B 6/04; A61B 6/03

(52) U.S. Cl. CPC A61B 6/589; A61B 6/035; A61B 6/0407; A61B 6/467; A61B 6/463; A61B 6/4417

(57) ABSTRACT

An imaging arrangement includes an imaging modality, a control facility, a moveable patient couch and a positioning apparatus. The positioning apparatus includes at least one optical image recording apparatus for recording at least one photo-realistic image of the patient couch. At least one item of positioning information is input with respect to an image of the patient couch shown on the display apparatus and a patient if applicable positioned thereupon. A method for positioning a patient in an imaging modality is further disclosed.

### PRIORITY STATEMENT

The present application hereby claims priority under 35 U.S.C. §119 to German patent application number DE 102014226756.0 filed Dec. 22, 2014, the entire contents of which are hereby incorporated herein by reference.

### FIELD

At least one embodiment of the invention generally relates to an imaging arrangement having an imaging modality, a control facility, a moveable patient couch and/or a positioning apparatus.

### BACKGROUND

If a radiological examination is to be performed for instance with a computed tomography system (CT), the body/organ area to be examined must be carefully selected and restricted to the area required diagnostically in order to avoid unnecessary radiation exposure. Nowadays the conventional method is to define the start point of a planned examination (diagnostic scan) via a laser light-beam localizer disposed in the scanning plane. With this method the patient lying on the CT couch is moved into the tunnel of the CT device by moving the couch until the light strip of the laser light-beam localizer is aligned with the desired start point of the examination area. During magnetic resonance examinations, this targeting is performed outside of the tomography system and the patient is subsequently moved so far into the tunnel of the tomography system that the selected examination area lies in the center of the tomography system.

Adjusting the couch position itself is typically performed by manually actuating control elements in order to move the couch in the couch longitudinal direction or if provided, in the vertical direction.

The display of the light strip on the patient in the gantry is the only visual feedback here for the person undertaking the planning. Here the knowledge relating to the planned examination and its parameters defined in the scanning protocol (body region/organ, starting position, scanning direction and length) is typically a verbal communication within a team and/or a recollection by the examining person undertaking the positioning.

The correctness of this positioning work is only apparent when the scan has been triggered and the result is made visible on the monitor as an emerging topogram or as an anatomical sequence of real-time images.

With the known procedures, there is the problem that the positioning cannot be performed on the console, where the knowledge relating to the examination to be planned and the defined scanning protocol is available, but instead directly on the couch in the examination room. Moreover, the positioning method is inaccurate if the position of the light strip of the laser light-beam localizer has to be estimated at a distance from outside of the tunnel. The positioning is only checked by way of the scan itself, as a result of which x-rays of the patient are already recorded in the case of a CT device.

### SUMMARY

Embodiments of the present invention specify an imaging modality and a corresponding positioning method, which allow for improved positioning.

At least one embodiment of the invention is directed to an imaging arrangement. At least one embodiment of the invention is directed to a method. An embodiment of the inventive method is preferably performed on an embodiment of the inventive apparatus. Advantageous developments of the invention form the subject matter of the claims.

With an embodiment of the invention, the position of the patient couch is detected by an optical image recording facility. This is preferably a 2D camera (photo or video camera). The image recording apparatus is configured such that it records an image of the top side of the couch (if applicable with the patient lying thereupon). An embodiment of the invention enables a photo-realistic graphical planning e.g. with start and end point of the examination area in conjunction with a current two-dimensional (2D) photo or video image of the patient on the patient couch. The representation and planning can be performed locally (on or in the visual range of the imaging arrangement) or remotely (on the console, e.g. in a control center) using a display apparatus, e.g. with a suitable touch screen on the gantry or any other interactive graphical input system. After planning and adjustment have taken place, the patient can be moved directly to the target position planned on the image of the patient on the couch, VIA one single movement command for instance.

An embodiment of the present invention is also directed to a method for positioning a patient couch supporting a patient in an imaging modality. This comprises:
placing a patient on a patient couch,
recording at least one image of the patient,
displaying the image on a display apparatus,
inputting at least one item of position information as a function of the image, and
positioning the patient by moving the patient couch into the imaging modality on the basis of the position information.

Embodiments of the method can be implemented here in the control apparatus as software or also as (permanently wired) hardware.

### BRIEF DESCRIPTION OF THE DRAWINGS

Further advantages, features and details of the present invention emerge from the description below of advantageous embodiments of the invention, in which:

FIG. 1 shows a computed tomography apparatus,

FIG. 2 shows a first image,

FIG. 3 shows a second image,

FIG. 4 shows a third image,

FIG. 5 shows a fourth image,

FIG. 6 shows a fifth image, and

FIG. 7 shows a flow diagram.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Further, although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

With an embodiment of the invention, the position of the patient couch is detected by an optical image recording facility. This is preferably a 2D camera (photo or video camera). The image recording apparatus is configured such that it records an image of the top side of the couch (if applicable with the patient lying thereupon). An embodiment of the invention enables a photo-realistic graphical planning e.g. with start and end point of the examination area in conjunction with a current two-dimensional (2D) photo or video image of the patient on the patient couch. The representation and planning can be performed locally (on or in the visual range of the imaging arrangement) or remotely (on the console, e.g. in a control center) using a display apparatus, e.g. with a suitable touch screen on the gantry or any other interactive graphical input system. After planning and adjustment have taken place, the patient can be moved directly to the target position planned on the image of the patient on the couch, via one single movement command for instance.

The image recording apparatus is preferably a (single) 2D digital camera (photo or preferably video camera), so that this provides solely two-dimensional, and not three-dimensional images. This does not actually allow positioning in the 3D space, but this problem is preferably overcome by the positioning apparatus being calibrated such that each point on the top side of the patient couch on the photo-realistic image can be assigned to a position along the longitudinal direction or to a position on the surface of the patient couch. An assignment to a position in the transverse direction of the couch can be provided, but is not decisive in the case of CT devices, since the examination area always extends over the entire scanning plane, which is aligned at right angles to the couch longitudinal direction. With magnetic resonance tomography systems on the other hand, the examination area (field of view) can be smaller and a positioning in both directions on the couch surface may be necessary.

The positioning information (e.g. start and end point of an examination) is therefore preferably input with respect to the image of the patient couch, wherein the examining person orientates him/herself e.g. to those areas that are not concealed by the patient or his/her clothing.

The planning on the image requires a clear coordinate transformation, i.e. a pixel in the planning area (on the image of the patient couch) must be clearly transformed into the coordinate system of the imaging modality, in simple terms, it must be possible to calculate which pixel of the displayed image corresponds to which couch coordinate. The couch coordinate is in turn known if absolute position sensors are used for the couch position in the imaging arrangement and the image recording apparatus is arranged in a fixed position.

For an empty and defined couch surface, the coordinate transformation is possible if the dimensions, surface and position of the empty couch in a functional system are known at any point in time and it is thus possible to calculate the spatial coordinate to which a pixel of the couch surface corresponds. If any three-dimensional object (patient) is however located on the couch, a three-dimensional detection of the modified object surface would be a requirement, in order to be able to perform such a transformation in the region of the three-dimensional object located on the couch. The invention solves this problem by performing the planning on the basis of the two-dimensional couch surface. In order to facilitate the input of positioning information, according to a preferred embodiment the positioning information is input with respect to an area of the image of the patient couch, which is not concealed by the patient, in particular with respect to the couch edge.

As a result, cost-effective 2D (video) cameras can be used in order to be able to perform a graphical planning of an examination area on the basis of a photo-realistic mapping of the patient on the couch on a suitable display.

The relative position of the patient couch compared to the imaging modality is if necessary calibrated once for each imaging arrangement. According to one embodiment of the invention, this is achieved by way of a suitable two-dimensional geometric structure (e.g. a checkerboard pattern), which is arranged at a precisely defined position on the surface of the empty couch. An image or video is then recorded by the image recording apparatus and a connection between the image or video pixels and the couch coordinate is established by way of a suitable algorithm, which identifies the geometric structure on the image/video. Advantageously this only needs to be performed once per installation of an imaging arrangement or only repeated when the camera is moved to another position. It is moreover advantageous here if the position detection of the patient couch is provided with an absolute position sensor, so that the connection between the image pixel/video pixel-couch coordinate-system coordinate system of the imaging arrangement, e.g. CT, only has to be calibrated once, and the position of the couch or the relevant points on the couch surface in the system are always known.

This also applies to imaging arrangements in which the height of the patient couch can be adjusted. On account of the absolute position sensor, the height of the couch surface relative to the calibrated position or the absolute distance to the image recording apparatus is known. A mapping rule can be determined therefrom e.g. by using the intercept theorem, said mapping rule assigning a position on the couch surface to each pixel on the image/video. The invention is however preferably applied to imaging arrangements without a vertical couch displacement.

At least one position marker can preferably overlay at least one image of the patient couch recorded with the image recording apparatus. The position marker is shown purely virtually here by it overlaying the representation of the patient couch on the display apparatus. This is advantageous in that it can always be identified, while for instance the clothing of the patient can cover an optical position marker. The embodiment of the position marker can also be changed arbitrarily, as a result of which it can be adjusted to the requirements of an examining person. A virtual strip of the couch edge is particularly preferably shown to the left and right by way of computer graphics. This virtual side strip corresponds by definition to the empty couch edge and is thus defined and can be calculated in respect of its coordinates like a completely empty couch. The virtual strip can preferably be shown/hidden depending on requirements. The position marker is preferably rasterized in the longitudinal direction, e.g. in cm steps. The alignment of the strips of the raster is preferably parallel here to the scanning plane of the imaging modality.

The virtual position markers shown, e.g. at the couch edges can then be used advantageously to perform a planning of the examination area. To this end, the examining person can easily interactively mark the desired examination area for instance (e.g. to the left or right) and define the direction, in other words the start and target coordinates across e.g. an interactive area beam and adjust the same if necessary. For the correctness of this method, it is important for the definition of the examination area only to take place on the virtual side edges shown, since it is only ensured here that the coordinates are always clearly defined and cannot be influenced by objects on the couch. According to a preferred embodiment, the image is indicated in color in the marked examination area, while the rest of the image outside of the marked examination area is shown in black and white.

The problem of absent feedback in respect of correct planning (according to a scanning protocol) can also be solved inter alia in that when a scanning or examination protocol is loaded, the examination parameters defined therein such as the scanning direction and length, examination area (field of view) and the selected body region/organ and type of examination are indicated on the display apparatus. In addition, indications of inconsistencies such as incorrect scanning direction or exceeding the possible scanning length or position can already be indicated during the active planning phase of the examination area. A check of the configured recording parameters can therefore be performed on the basis of an image recorded with the image recording apparatus. An error message can advantageously be output if an item of impermissible position information input is identified.

In particular, an incorrect scanning direction and/or the exceeding of a permissible examination area can be output as impermissible recording parameters. Moreover, checks can also be performed as a function of the loaded scanning or examination protocol to determine whether the positioning of the patient is optimized for the organ to be examined. If the examining person makes a mistake during the manual input of the examination area, be it as a result of inadequate experience or lack of concentration, the position input is also checked in this respect. A check and if applicable adjustment of the planning is therefore possible even before triggering the radiation.

The image recording facility is preferably arranged such that it detects an area in front of or behind the imaging modality. It can be arranged on or above the imaging modality, e.g. fastened to the ceiling or in the case of a CT device to the gantry.

A patient positioned on the patient couch can preferably be detected. The position of the patient relative to the patient couch and the position of the patient couch in comparison to the imaging modality can then also be detected. This allows conclusions to be drawn overall as to the position of the patient relative to the imaging modality.

The positioning apparatus can particularly advantageously have a storage unit and a display apparatus, wherein at least one item of positioning information can be input on the basis of an image of the patient couch and/or of the patient shown on the display apparatus. The ability to input the positioning information by way of the display apparatus provides for a very exact and reproducible positioning. The positioning information can also be stored and is thus available for checks of the examination performed.

Advantageously the display apparatus can be embodied as a touchscreen apparatus and an item of positioning information can be input by touching the display apparatus. The display apparatus can therefore be embodied as a tablet or ultrabook (a notebook with touchscreen). Alternatively, it is naturally possible to input the positioning information on any type of computer using a mouse or via keyboard entries, for instance arrow keys. Any other interactive graphics system can also be used.

Advantageously the display apparatus can be arranged on the imaging modality. Alternatively, the display apparatus can be arranged in a control room, e.g. on a console from which the imaging modality is checked. With this latter arrangement, the examining person need not be present in the examination area, but instead after positioning the patient on the patient couch the entire examination can be performed from a control room.

According to one embodiment, at least one optical and/or modality-specific physical (not virtual) position marker can be arranged on the patient couch. The optical position marker can be embodied as a raster at the edge of the patient couch. The couch edges can however also be provided with grids or other visually detectable representations. For a modality-specific physical position marker, with a computed tomograph this may be a metallic element and with a magnetic resonance system this may be a water-filled volume. A geometrically delimited area is therefore generated in an image of the imaging modality, said area not generating any signal or generating a higher-than-average signal and as a result contrasting from the rest of the image. The modality-specific position marker is preferably also an optical position marker. The image of the image recording facility and an image of the imaging modality can then be aligned.

The imaging modality can preferably be embodied as a computed tomography apparatus. In more general terms or alternatively the apparatus can be embodied as an imaging modality with a hollow cylindrical patient receptacle, e.g. a magnetic resonance system, PET or SPECT system.

An embodiment of the present invention is also directed to a method for positioning a patient couch supporting a patient in an imaging modality. This comprises:
placing a patient on a patient couch,
recording at least one image of the patient,
displaying the image on a display apparatus,
inputting at least one item of position information as a function of the image, and
positioning the patient by moving the patient couch into the imaging modality on the basis of the position information.

The examination or the scan is then preferably performed according to the position information input.

Further advantageous embodiments of the inventive method correspond to corresponding embodiments of the inventive imaging modality. In order to avoid unnecessary repetitions, reference is thus made to the corresponding apparatus features and their advantages.

An actuation button for moving the patient couch may preferably be present and the patient couch can be moved by way of a predetermined actuation movement of the actuation button. For instance, a single tap on the actuation button is sufficient to move the patient couch into the scan starting position (target position) on the basis of the position information input.

According to a preferred embodiment, a live video stream recorded by the image recording apparatus is shown on the display apparatus while the couch is moved into the scan starting position in order to render visible in the display the side of the couch facing away from the examining person (patient side facing away), in order if applicable to be able to intervene if any pipes or cables become jammed along the couch's movement path. After the target position has been reached, the patient image which is frozen at the start of the movement preferably appears for further actions if necessary, e.g. planning and positioning for further examinations.

The afore-cited methods can be implemented here in the control apparatus as software or also as (permanently wired) hardware.

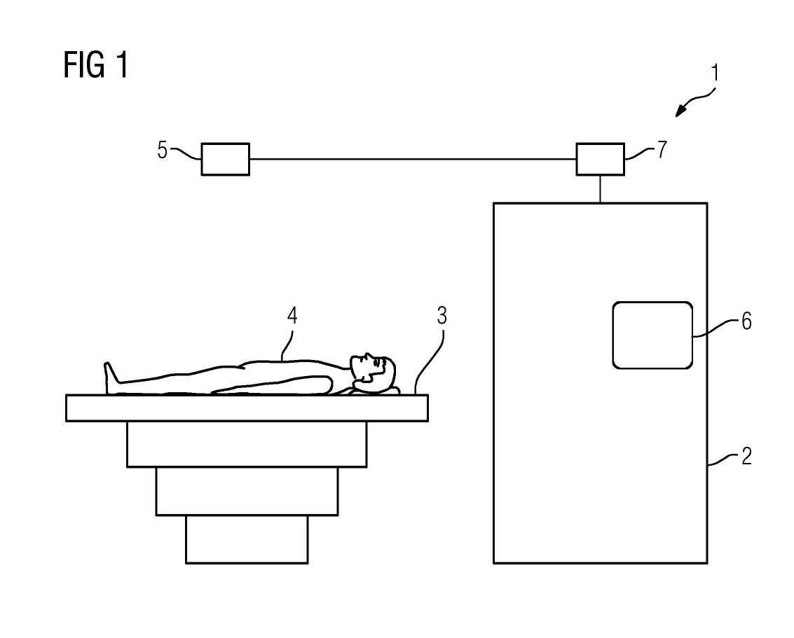

FIG. 1 shows an imaging arrangement 1 with a computed tomography apparatus 2 having a hollow cylindrical patient receptacle. A patient couch 3, upon which a patient 4 rests, can be moved into the computed tomography apparatus 2. A digital camera 5 is arranged in a fixed position above the patient couch 3, e.g. fastened to the ceiling, with which an area in front of the computed tomography apparatus 2 can be detected. In particular, the top side of the patient couch 3 can be mapped.

A display apparatus 6 is arranged on the computed tomography apparatus 2. The computed tomography apparatus 2, the digital camera 5 and the display apparatus 6 are connected by way of a control apparatus 7.

The display apparatus 6 is embodied as a tablet, also referred to as a tablet computer or as a notebook. The display apparatus 6 accordingly comprises a touchscreen 8. The display apparatus 6 is thus simultaneously an input apparatus.

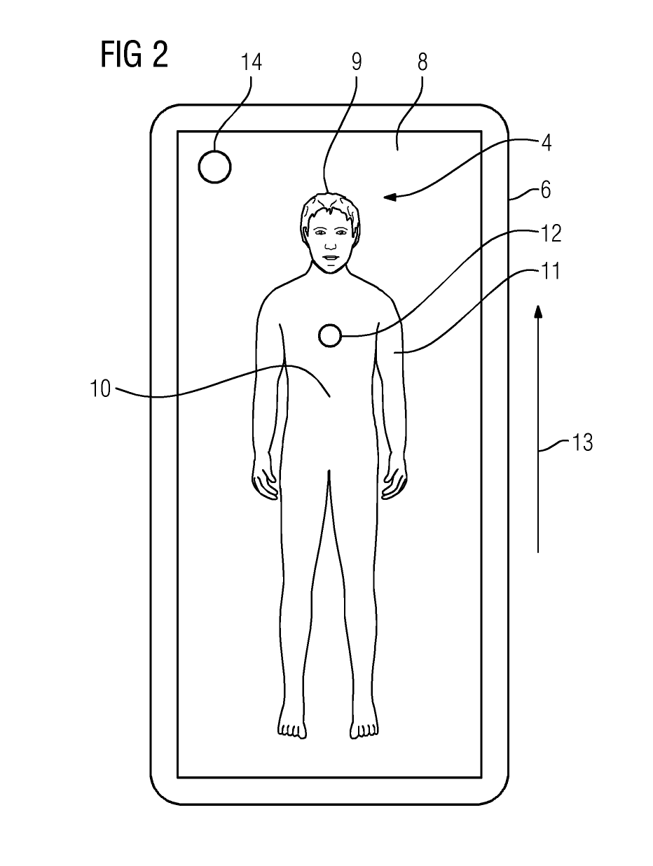

FIG. 2 shows the display apparatus 6 in detail. The patient 4 resting on the patient couch 3 or at least one part captured by the digital camera 5 can be presented photo-realistically e.g. in a video representation, as shown in the figures below.

FIG. 2 shows the head 9, the torso 10 and the arms 11. Depending on the examination in question, the examining person touches a point 12 on the touchscreen 8, as a result of which a position corresponding to this point 12 is predetermined in the longitudinal direction, in other words in the direction in which the patient couch 3 can be moved. The examining person preferably selects the point 12 on a part of the image, which corresponds to part of the patient couch 3, in which its surface is not concealed by the patient 4, since the image is calibrated to the surface of the patient couch. This need not be prescribed however so that an input of a point 12 at any point on the touchscreen is accepted. The point 12 marks the start point of a scan. The examining person is then possibly requested to input a further point (not shown), which marks the end point of a scan and thus defines the entire examination area. Alternatively, the end point is predetermined by the scanning protocol already defined. The scanning direction is shown by the arrow 13 and may possibly likewise be changed by an input on the display apparatus 6.

The relative position between the area 12 and the computed tomography apparatus 2 can be concluded from the known relative position between the digital camera 5 and the computed tomography apparatus 2.

Accordingly, the control apparatus can trigger a movement of the patient couch 3 in the longitudinal direction, so that the area 12 is positioned in the center of the computed tomography apparatus 2 and thus represents the start point of the scan or of the examination.

The actuation button 14 for starting the patient couch can be embodied as a predetermined area on the touchscreen 8 or in more general terms on the display apparatus 6. The patient couch 3 is then moved by touching the touchscreen at this point.

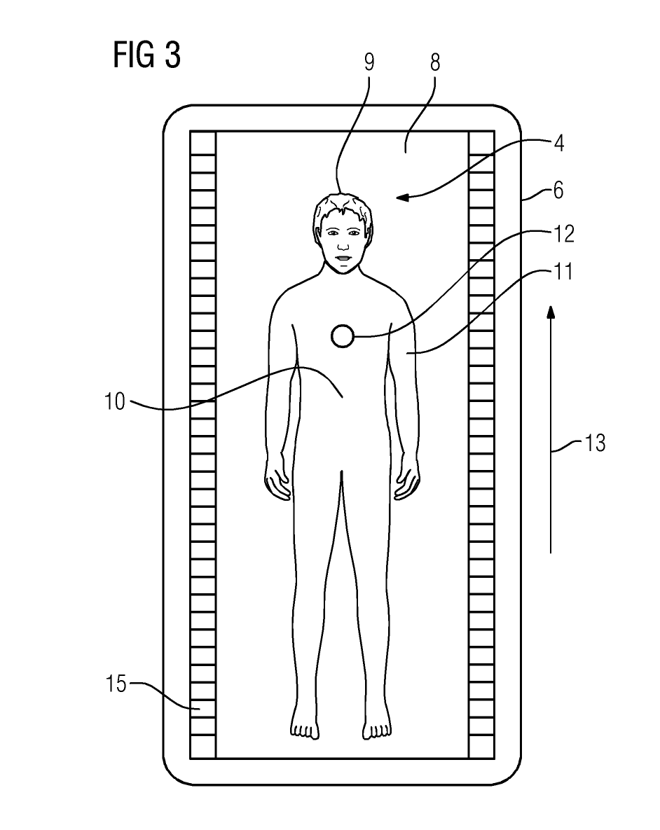

FIG. 3 shows a further display option of the patient couch 3. A raster 15 is superimposed here onto the couch edges as a position marker in each case. This serves to improve visualization of the surface of the patient couch 3 to which the positioning apparatus is calibrated. Since the image recording apparatus does not supply a 3D image, an exact positioning on the patient 4 him/herself is not possible since this contrasts three-dimensionally from the couch surface. However if the examining person has a reference point on the couch surface, here the position marker, he/she can estimate relatively precisely from the perspective representation of the patient which course an (imaginary) scanning plane, in other words a plane at right angles to the couch longitudinal direction, will be taken by the patient. With the aid of the position marker the examining person can orientate him/herself to the couch edge, even if this is entirely or partially concealed by the patient's clothing. This allows for a more accurate positioning of the examination area.

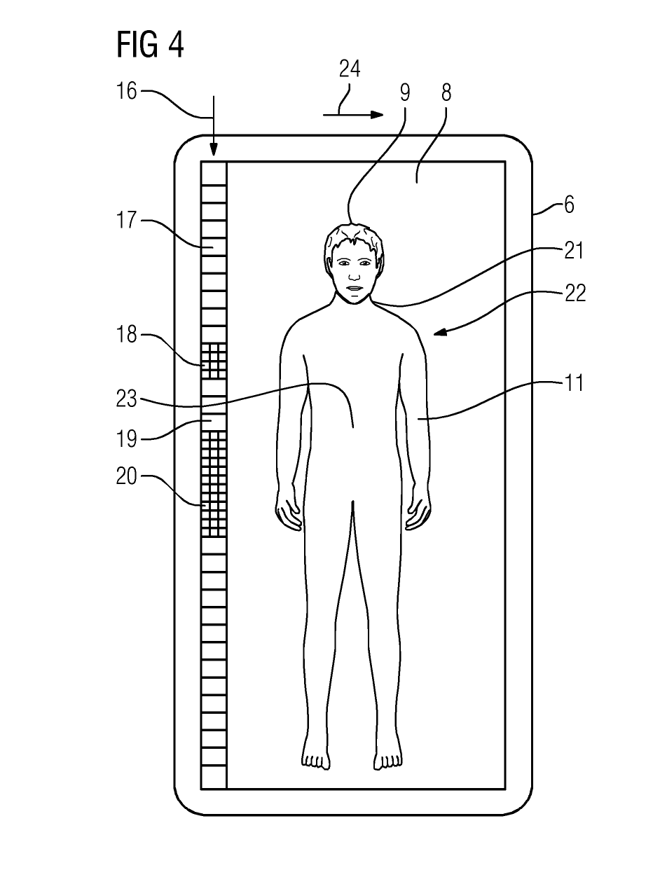

The position marker 16 can preferably contain examination area-specific details, as FIG. 4 shows. For instance, a first area 17 of the position marker can be rasterized, a second area 18 can have a grid, and this sequence can be repeated in all further areas 19 and 20. The first area 17 reproduces e.g. the extension of the head 9, the second area 18 that of the heart 21, the third area 19 that of the abdominal region 22 and the fourth area 20 that of the hips 23 of the patient 4.

The areas 17 to 20 can also be contrasted from one another by way of different coloring or other optical distinction aids. Their number is basically arbitrary and can be adjusted to the mapped region of the patient 4 or examination conditions.

A further position marker can also be indicated in the direction of the arrow 24, but it is only required if a displacement of the patient couch 3 in this direction is also possible.

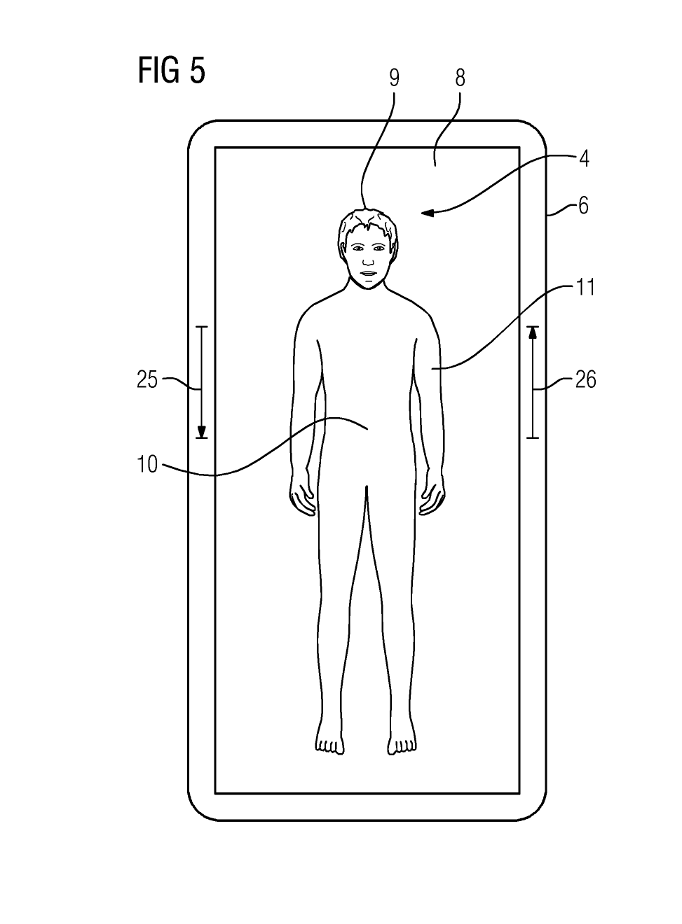

FIG. 5 shows a further embodiment of the input of the examination area. Instead of tapping as shown in FIG. 2, a desired segment in the longitudinal direction of the couch is swiped over by the examining person, for instance one of segments 25 and 26. At the same time the scanning direction can be predetermined by taking account of the direction swiped over. In this way, it is not only the center that can be defined as the middle of the swiped-over area 25 or 26, wherein the center of the examination area is aligned with the center, in other words the middle point in the axial and/or radial direction, of the computed tomography apparatus 2, but instead also the area to be mapped, also referred to as examination area or field of view (FOV).

This type of input of the examination area can be particularly advantageously combined with the superimposed position marker as shown in FIG. 4. In particular, the selection of a field of view can then be defined by tapping one of the areas 17 to 20.

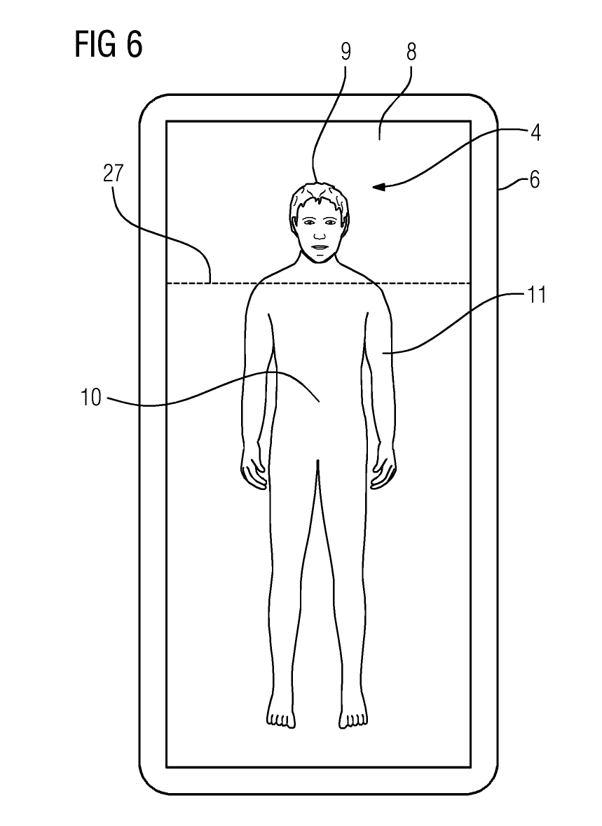

FIG. 6 shows a further embodiment of the representation of the examination area. Here the position of the laser light strip and thus of the center of the computed tomography apparatus 2 is shown superimposed on the patient 4 as a line 27 on the display apparatus 2. Since it is only shown, it cannot adjust to the contour of the patient like the light strip of a light-beam localizer, but the examining person can nevertheless approximately visualize the line course with the aid of the image (photo or video) of the patient. For positioning purposes the examining person should orientate him/herself to the course of the line shown on the couch edge, which is not concealed by the patient, since the line 27 is calibrated to the surface of the couch. The line 27 moves with the movement of the patient couch. As a result, after concluding the movement of the patient couch 3, the examining person can check whether the position input has taken place as desired. The line 26 is at right angles to the movement direction of the patient couch 3 and with a single movement in the longitudinal direction is at right angles to the longitudinal direction of the patient couch 3.

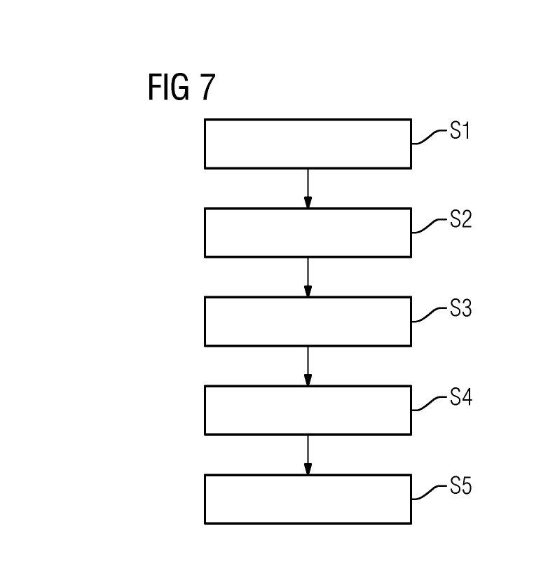

FIG. 7 shows a flow diagram for a method for positioning a patient couch 3 supporting a patient 4 in an imaging arrangement.

In step S1, the patient 4 is placed on a patient couch 3. In the following step S2, at least one image is recorded with an optical image recording apparatus 5. With the aid of this image, it is possible to determine the position of the patient 4 in comparison to the top side of the couch 3, since the relative position of the digital camera 5 in respect of the C top side of the couch 3 is known.

The image is then shown on the display apparatus 6 as step S3. A position marker 16 is in particular superimposed onto the patient image.

Position information is then input onto the display apparatus 6 in step S4 as a function of the image, either by touching the image or by swiping over a segment 25 or 26. Here the display apparatus 6 can be in a position input mode, which can be activated for instance by pressing a specific button. In other words, a position input cannot always occur but only if, by pressing the button, the display apparatus 6 expects a position input.

Then in step S5, the patient 4 is positioned in the computed tomography apparatus 2, taking into account the position information.

With all example embodiments, an image is understood to mean in particular also a video image, i.e. that the individual images are available in real-time and can form the basis of the input of a position marker.

At least one embodiment of the invention thus allows for a simple and intuitive planning on a photo-realistic basis by computer graphics without hardware indicators for the examination area additionally being required on the couch hardware. No 3D contour detection of the patient located on the couch is thus necessary. By planning and possibly showing a position marker on the virtual couch edge, reliable planning is always possible, irrespective of whether the real couch edge is covered. Moreover, an unnecessary radiation exposure can be avoided particularly with computed tomography apparatuses.

The aforementioned description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods. Further, elements and/or features of different example embodiments may be combined with each other and/or substituted for each other within the scope of this disclosure and appended claims.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Further, at least one embodiment of the invention relates to a non-transitory computer-readable storage medium comprising electronically readable control information stored thereon, configured in such that when the storage medium is used in a controller of a magnetic resonance device, at least one embodiment of the method is carried out.

Even further, any of the aforementioned methods may be embodied in the form of a program. The program may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

None of the elements recited in the claims are intended to be a means-plus-function element within the meaning of 35 U.S.C. §112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An imaging arrangement, comprising:
an imaging modality;
a control facility;
a moveable patient couch; and
a positioning apparatus, including at least one optical image recording apparatus to record at least one image and a display apparatus to display the recorded image, the at least one optical image recording apparatus being arranged in at least one position of the moveable patient couch above the movable patient couch and being configured to record a photo-realistic image of the movable patient couch and a patient if applicably positioned thereupon, wherein at least one item of positioning information is imputable with respect to an image of the movable patient couch shown on the display apparatus and the patient if applicably positioned thereupon.

2. The imaging arrangement of claim 1, wherein the positioning apparatus is calibrated such that each point on the top side of the movable patient couch on the photo-realistic image is assignable to a position along the longitudinal direction of the movable patient couch.

3. The imaging arrangement of claim 1, wherein the positioning information is input with respect to an area of the image of the movable patient couch and the patient positioned thereupon which is not concealed by the patient.

4. The imaging arrangement of claim 1, wherein the positioning information is input with respect to a position marker, shown superimposed onto the image shown of the patient.

5. The imaging arrangement of claim 4, wherein the position marker represents part of the top side of the movable patient couch.

6. The imaging arrangement of claim 1, wherein the display apparatus is embodied as a touchscreen, and an item of positioning information is imputable by touching the display apparatus.

7. The imaging arrangement of claim 1, wherein the display apparatus is arranged on the imaging modality.

8. The imaging arrangement of claim 1, wherein the display apparatus is arranged in a control room.

9. The imaging arrangement of claim 1, wherein the image recording apparatus is embodied as a 2D digital photo camera or 2D digital photo camera.

10. The imaging arrangement of claim 1, wherein the imaging modality is embodied as a computed tomography apparatus.

11. The imaging arrangement of claim 1, wherein certain parameters of a selected examination protocol are showable on the display apparatus superimposed over the image of the patient couch.

12. A method for positioning a patient couch supporting a patient in an imaging modality, the method comprising:
recording at least one image of the patient, located on the patient couch, with an optical image recording apparatus;
representing the image on a display apparatus;
receiving at least one item of position information input as a function of the image; and
positioning the patient by moving the patient couch into the imaging modality based upon the received at least one item of position information.

13. The method of claim 12, wherein certain parameters of a selected examination protocol are displayed on the display apparatus, superimposed with the representation of the image.

14. The method of claim 12, wherein the at least one item of position information is checked for inconsistencies and if applicable, a fault message is output before the patient is positioned by moving the patient couch.

15. The method of claim 12, wherein an actuation button is present for moving the patient couch and wherein the patient couch is movable by an actuation movement of the actuation button.

16. The method of claim 12, wherein the imaging modality is embodied as a computed tomography apparatus and the at least one item of positioning information comprises at least the start and end point of the scanning area.

17. The imaging arrangement of claim 5, wherein the position marker represents one or a number of segments at the edge of the movable patient couch.

18. The imaging arrangement of claim 2, wherein the image recording apparatus is embodied as a 2D digital photo camera or 2D digital photo camera.

19. The imaging arrangement of claim 2, wherein the imaging modality is embodied as a computed tomography apparatus.

20. The imaging arrangement of claim 11, wherein certain parameters of the selected examination protocol are the scanning direction and the scanning length.

21. The method of claim 13, wherein the at least one item of position information is checked for inconsistencies and if applicable, a fault message is output before the patient is positioned by moving the patient couch.
